(19) United States
(12) Patent Application Publication
(10) Pub. No.: US 2022/0183897 A1
(43) Pub. Date: Jun. 16, 2022

(54) Medical Device For Wound Care Sealing Applications and Method of Using Same

(71) Applicant: Semech, LLC, Roswell, GA (US)

(72) Inventors: Christopher G. McDowell, St. Augustine, FL (US); Sirka Hummel, Casper, WY (US)

(21) Appl. No.: 17/686,383

(22) Filed: Mar. 3, 2022

Related U.S. Application Data
(63) Continuation of application No. PCT/US2020/049287, filed on Sep. 3, 2020.
(60) Provisional application No. 62/894,969, filed on Sep. 3, 2019.

(51) Int. Cl. A61F 13/02; B25G 3/02

(52) U.S. Cl. CPC A61F 13/0236; A61F 5/443; B25G 3/02

(57) ABSTRACT

A medical device and methods of applying a barrier product are described that allow for improved adhesion of a barrier product to skin in the area of a wound such as a stoma, fistula or other opening in human skin. The medical devices herein include a handle; a roller arm; and a roller head. The exterior surface of the roller head is configured to enhance adhesion of a barrier product to an area of mammal skin near a wound opening. Kits and other options are described for facilitating use of the medical devices by patients having wounds or medical professionals.

### CROSS-REFERENCE TO RELATED APPLICATIONS

This Application is a U.S. Continuation Nonprovisional Application of International Patent Application No PCT/US2020/049287, filed, Sep. 3, 2020 and designating the United States, which application claims the benefit under 35 U.S.C. § 119(e) to U.S. Provisional Patent Application No. 62/894,969, filed Sep. 3, 2019, entitled “Medical Device For Wound Care Sealing Applications and Method of Using Same,” the entire disclosures of which are incorporated herein by reference.

### BACKGROUND OF THE INVENTION

### Field of the Invention

The invention relates to the field of wound care and the application of wound care dressings that employ barrier seals, barrier seal compounds and other wound care adhesives and appliances associated with such barrier seals, barrier seal compounds and other wound care adhesives for use on an open wound into the body, particularly as may be associated with a stoma, fistula or other open body wound, whether such wound originates surgically or non-surgically.

### Description of Related Art

A significant number of people worldwide currently have, or have had a medical condition in which either a temporary, or permanent stoma has resulted. A stoma is an opening which is typically surgically created in the body (e.g., in the colon, ileum or even in the urinary tract) to divert fecal matter or urine in patients. Stomas may be necessary when the bowel or bladder is obstructed, the bowel is perforated or when a section of the bowel or bladder requires removal due to disease. Such situations occur, for example, due to bowel or bladder cancer, diverticular issues or diseases, ulcerative colitis, Chron's disease, urinary tract disease or the like. The stoma allows fecal matter or urine leaving the stoma in the form of effluent to be collected in a drainage pouch (e.g., an ostomy bag). The patient having the stoma is referred to medically as an ostomate.

Additionally, some patients have wounds which, while not formed as a stoma, may also exhibit release of effluent as a result of drainage or leakage of fluids. Such wounds may result from surgery, trauma, infection, or other etiology in which a large opening exists into the body through the skin, requiring a medical dressing as a cover and/or to manage healing of the wound.

A fistula is a further medical condition which calls for a medical dressing similar to those used for treating a stoma. A fistula is an abnormal connection between an organ, vessel, or intestine and another structure, typically the skin. The fistula prolapses through the skin, similar to a stoma in appearance and behavior. Fistulae can arise due to illnesses, trauma or surgery and require wound care and treatment using similar wound dressings to those used for a stoma or other open wound.

It is estimated that approximately 120,000 new stomas will be created in the United States each year, and one million patients will have stomas at any given time in North America. See, S. Husain et al, “Late Stomal Complications,” Clin. Colon Rectal Surg. (Feb. 2008) Vol. 21(1), pp. 31-40. Others also predict that the number of ostomates will continue to increase by 3% annually as stoma creation is routinely performed by a diversity of surgical subspecialists. See, G. B. Turnbull, “Ostomy Statistics: The $64,000 Question,” Ostomy Wound Manage. (2003), vol. 49(6), pp. 22-23.

Complications are common when using ostomy appliances and wound dressings for use in dressing or maintaining a patient's stoma, fistula or other wound, as are issues associated with the wound care appliance's modes or the means of attachment of the associated dressing that can lead to leakage of stool, fluid effluent, and/or urine. Such leakage or device detachment can result in significant health consequences such as infections, frequent doctor or hospital visits, and downstream medical consequences as well as taking a psychological toll on the ostomate.

The primary complication for ostomates and other wearers of similar dressings and appliances is leakage caused by improper attachment, user error or other issues (such as wrinkled or uneven skin, skin trauma or lack of wound care maintenance). Studies have shown that leaking does not necessarily decrease over time or with additional experience in appliance or wound care management.

Prior art attempts to address such problems are generally directed to attempts to improve pouch attachment for ostomy pouches or other wound care bags and appliances, either through their design, or through the use of various adhesives, barriers, barrier seals and associated adhesive compounds available to patients for use with their dressings and ostomy pouches or other wound care appliances. Materials selected for such care depend on the characteristics of the stoma or fistula and may include materials such as skin barriers, adhesives, dressings, pouches and wound managers, as well as negative pressure dressings such as vacuum-assisted closures. See, R. Hoedema, M. S., “Enterostomal Therapy and Wound Care of the Enterocutaneous Fistula Patient,” Clin. Colon Rectal Surg. (Sept. 2010), vol. 23(3), pp. 161-168.

Skin protection is a primary concern for ostomates and other wound care patients as the opening in the skin may have varying outputs that may be acidic, alkaline, or contain proteolytic enzymes, all of which may damage skin. Id. Barriers are a primary method for protecting the skin surface from effluent. Such barriers include solid wafers, powder, paste, and other sealants. Id.

Some of the products, such as wafers, are pectin-based, and have their own adhesive surface which can melt on contact with the patient's skin to form a seal. The wafers ultimately need to be changed or can fail due to melting. Powders are used for wet or weeping wounds, and may be pectin or karaya-based to absorb moisture. Management of the amount and application of such materials on shaved skin can be variable and may not be useful long term. Barrier pastes can also be used as an edge reinforcement for wafers and pastes can be applied to smooth out the skin. Skin surface irregularities or folds contribute to difficulties encountered in using various wound care dressings and products.

Use of barrier devices are indicated for longer term wound care. There are available liquid and aerosol adhesives as well as double-faced adhesive sheets or discs as noted above that can serve as barrier seals. Id. Dressings are also used as cost-effective wound care for simple and low effluent stoma or fistulas. Gauze dressings can also be used with skin barriers for wound management in such situations. But for higher effluent situations, generally a pouch or other bag system are employed to avoid issues related to skin trauma and allow for effluent monitoring. Id. Skin irregularities are known to contribute to ineffectiveness in barriers, as many times adhesives are employed with such barriers to assist such products or simply for direct attachment. Irregularities include folding or wrinkling of skin or body folds and contours and can make forming an effective seal difficult.

Seals and seal barriers, which can include hydrocolloid or other similar chemical compounds or solvents are generally applied pen-wound, i.e., to the surface of the skin around the wound, as part of stoma, fistula or other wound dressing management solutions as noted above.

Current techniques used to apply such wound care compounds to irregular skin and body shape contours involve use and application of either pre-cut, or cut-to-fit and mold-to-fit sealing materials. Such materials are manually applied by hand using the pressure of an applicant's fingertips or hands to mold such materials into a desired position on the patient and around the wound. Because the barrier seal, barrier seal compound and/or adhesive is applied and placed manually using pressure from the fingers or hands, adhesion of the compound or seal to the skin's surface is not generally fully cohesive, and can leave moderate areas lacking full surface-to-surface interfacial contact around the wound, stoma, fistula, or identified wound's protrusion or location.

The surfaces of a human body naturally contain folds, wrinkles and other irregularities, as noted above, including deformities, scars, moles, and other protrusions or indentations. Barriers or other adhesive devices applied peri-wound when being adhered to such irregular surfaces and body contour, can create a strain in bringing the adhesive into interfacial contact between the barrier or other device or compound and the skin surface.

Stress in the sealing agent's or dressing's ability to adhere can often exceed the holding power of the associated adhesive, leading to a compromise in the ability to retain the dressing and/or other wound care appliances, such as an ostomy pouch, to the skin. In such situations, the barrier or pouch may separate from the skin. This can create significant problems in areas of post-surgical skin swelling or skin trauma and is expected when there are skin irregularities and contours. Such separation leads to effluent leakage under and around the barrier, which is a significant problem for the vast majority of ostomates.

Hydrocolloid seals and skin barriers that include hydrophilic particles or absorbents, used for moisture absorption can transmit moisture when conditions are saturated. This can cause additional pulling and weighing down on an appliance, and also impact the adhesive either through swelling, loss of cohesion and/or disintegration, leading to leakage and its associated issues.

Commonly and widely practiced methods used by healthcare professionals and patients alike to increase and secure adhesion, and try to achieve sufficient contact between one of the above-noted barrier compounds or adhesives and the skin include the use of the tips of a pair of scissors, the use of the tips of tongue blades, or the use of the tips of a pair of hemostats which are each pressed into the compound in multiple areas. This is intended to create multiple areas of micro-indentation around the perimeter of the applied barrier or other compound once it is on the skin, and to mold it into the correct areas. This is not a formalized or standardized medical instrument or device that is created for stoma, fistula or other open wound care use, but simply a technique that has developed to address issues arising from manual application using ones' hands or fingers.

A further commonly used practice is the use of a hair dryer in hospitals, operating rooms, healthcare facilities and home care. The hair dryer is used on the barrier seal adhesive prior to adhering it to the patient's body or to another substrate. Such hair dryer use introduces a risk of infection by spreading bacteria and microorganisms, as well as potential injury through inadvertent physical contact with the hair dryer which can impact the wound or burn the patient's skin. It is also a time consuming technique.

Such techniques noted above are wide-spread in hospitals, emergency rooms, operating rooms, healthcare facilities and home care worldwide. Use of such techniques including the use of medical instruments like scissors have also been incorporated as part of teaching methods employed by various wound care nurses and ostomy specialists. Such methods can be seen in professional marketing “how to” videos, and in teaching documents, and in the “tips and tricks” sections of various ostomy websites, magazines and articles. These techniques unfortunately present a number of risks to the patient as noted above.

Further, the use of medical instruments, like hair dryers, is unsafe for several reasons, including an increased risk of infection, the spreading of bacteria or microorganisms from patient-to-patient should the implement be used repeatedly and not be a pre-sterilized medical instrument, and the risk of injury. Tongue blades can splinter or break and enter into a stoma, fistula or wound. Scissors tips can be sharp, and should the applicant slip, can result in a cut or other injury to the stoma, injury or wound or the surrounding pen-wound skin.

Based on the foregoing, there is a need in the art for an improved method of application and/or of wound care associated with stomas, fistulae and other open patient wounds with the goal of decreasing the percentage of ostomates and other patients who experience leaking, appliance failure or improper dressing application or replacement. There is further a need in the art not only for an improved method of dressing such wounds so as to prevent leakage, but also for an application technique that avoids use of unsafe techniques that have developed to address leakage, adhesion and detachment issues arising from application of wound care dressings, such as barrier seals and barrier seal compounds and adhesives to skin irregularities and contours by way of sharp implements, hair dryers and the like.

### BRIEF SUMMARY OF THE INVENTION

The invention includes a medical device that comprises a handle; a roller arm; and a roller head, wherein an exterior surface of the roller head is configured to enhance adhesion of a barrier product to an area of mammal skin near a wound opening.

In one embodiment, the handle comprises: a handle body having a proximal end and a distal end; a handle nozzle on the distal end of the handle body, wherein the handle nozzle is configured to receive a roller arm attachment on the roller arm. The roller arm attachment may be positioned on a proximal end of the roller arm. The handle body and the handle nozzle are preferably optionally releasably attachable. The handle body may define a hollow interior space for receiving wiring and/or electronics. The handle body may also have a removable inner sleeve surrounding the interior space. The proximal end of the handle body in one embodiment may have a removable end cap thereon.

The roller arm attachment may comprise an electrical connector. Further, the handle body may define a hollow interior space for receiving wiring and/or electronics, and the electrical connector may be a female connector configured to receive a male connector in electrical communication with the electronics positioned within the hollow interior space of the handle body.

If electronics are provided, they may include one or more of a heating element, a lighting element, and/or a motor. The handle nozzle may have a proximal end configured to connect to the distal end of the handle body, and the handle nozzle may have a distal end having a distal end portion configured to mate with the attachment on the roller arm, wherein the distal end of the handle nozzle may then further comprise an electrical connector for engaging an electrical connector on the attachment on the roller arm. If the electrical connector is placed on the distal end of the handle nozzle, it may be a male connector and the electrical connector on the attachment on the roller arm may then be a female connector. The handle may have an exterior surface configured for manual gripping.

The roller arm may comprise: an angled roller arm body extending from a connector end to a mounting end. In one embodiment, the roller arm body is configured to extend from the connector end and bend around the roller head so that the mounting end fits within an opening in the roller head. The roller arm body may also comprise a connector configured to mate with the handle nozzle on a first side of the connector and to engage a first longitudinally extending portion on a second side of the connector, a first bend extending between the first longitudinally extending portion and an angled portion, a second bend extending between the angled portion and a first transverse portion, a third bend between the first transverse portion and a second angled portion; and a fourth bend extending between the second angled portion and a mounting portion. The handle body and the handle nozzle may be formed as a unitary single piece body. The roller head may comprise protrusions arranged in a staggered pattern on the exterior surface of the roller head. The roller head also may comprise protrusions extending from the exterior surface of the roller head and having a blunt tip. The roller head may comprise protrusions extending from the exterior surface of the roller head and having a rivet-shaped tip. The roller head preferably has an opening on at least one side thereof for receiving a mounting portion of the roller arm. Preferably, the roller head is removable from the roller arm.

The invention also includes a medical device kit, comprising a medical device, comprising a handle; a roller arm; and a roller head, wherein an exterior surface of the roller head is configured to enhance adhesion of a barrier product to an area of mammal skin near a wound opening; and instructions for using the medical device. The kit may further comprise one or more additional and interchangeable roller heads. The kit may also comprise a barrier product. The barrier product preferably comprises a barrier and a barrier compound, and instructions.

The invention further includes a method of applying a barrier product for wound care, comprising: (a) providing a medical device, comprising a handle; a roller arm; and a roller head, wherein an exterior surface of the roller head is configured to enhance adhesion of a barrier product to an area of mammal skin near a wound opening; (b) applying the barrier product to an area of mammal skin near a wound opening so as to adhesively contact the area of the mammal skin near the wound area; (c) manually contacting the roller head to the barrier product and rolling the roller head across the barrier product in the area of the mammal skin near the wound area. In the method, the roller may comprise blunt protrusions on the exterior surface of the roller head, and the method may further comprise contacting the protrusions with the barrier product. The mammal skin is preferably human skin, the area near the wound may be a peri-wound area, and the barrier product may comprise a barrier compound, and the method may then further comprise applying the barrier compound to the pen-wound area and roller head may be rolled around the peri-wound area. The roller head is rolled in a one-way direction, a back-and-forth direction or a circular direction. The medical device may comprise a heating element and the method may further comprise heating the roller head prior to contacting the roller head to the barrier product.

The invention also includes a method for increasing adhesion of a barrier product to skin of a mammal in an area near a wound, comprising (a) providing a medical device, comprising a handle; a roller arm; and a roller head, wherein an exterior surface of the roller head is configured to enhance adhesion of a barrier product to an area of mammal skin near a wound opening; (b) applying the barrier product to an area of mammal skin near a wound opening so as to adhesively contact the area of the mammal skin near the wound area; and (c) manually contacting the roller head to the barrier product and rolling the roller head across the barrier product in the area of the mammal skin near the wound area.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a front perspective view of one embodiment of an medical device according to the invention showing a unitary handle body, handle body nozzle and lift arm attachment;

FIG. 2 is a perspective side elevational view of a medical device according to a modified embodiment of the medical device of FIG. 1 with the exception that the handle body, handle body nozzle and lift arm attachment are releaseably attachable to one another;

FIG. 3 is a is a front perspective view showing the distal end of the handle body nozzle of FIG. 2;

FIG. 3A is a front end elevational view of the handle body nozzle of FIG. 3;

FIG. 3B is a side elevational view of the handle body nozzle of FIG. 3;

FIG. 4 is a front perspective view of the roller arm of the medical device of the embodiment of FIG. 2;

FIG. 4A is a top elevational view of the roller arm of FIG. 4;

FIG. 5 is a rear perspective view of the handle body of the medical device embodiment of FIG. 2;

FIG. 5A is a front end elevational view of the handle body of FIG. 5;

FIG. 5B is a side elevational view of the handle body of FIG. 5;

FIG. 6 is a front perspective view of a roller head having rivet-shaped protrusions for use in with as an interchangeable roller head in the embodiment of a medical device shown in FIG. 2;

FIG. 6A is a side elevational view of a mounting side of a roller head having blunt end protrusions as shown in the medical device embodiment shown in FIG. 2;

FIG. 6B is a front elevational view of the roller head of FIG. 6A;

FIG. 7 is a front perspective view of an end cap for use in the embodiment of a medical device embodiment shown in FIG. 2;

FIG. 7A is a side elevational view of the end cap of FIG. 7;

FIG. 7B is a front elevational view of the end cap of FIG. 7;

FIG. 8 is a prospective view of an alternate embodiment of the medical device herein;

FIG. 9 is an exploded view of the medical device of FIG. 8;

FIG. 10 is a perspective view of a roller head of the device of FIG. 8;

FIG. 10A is a side elevational view of the roller head of FIG. 10;

FIG. 10B is an end elevational view of the roller head of FIG. 10;

FIG. 11 is a perspective view of a roller end cap of the device of FIG. 8;

FIG. 11A is a side elevational view of the roller end cap of FIG. 11;

FIG. 11B is a front elevational view of the roller end cap of FIG. 11;

FIG. 12 is a perspective view of a roller arm of the medical device of FIG. 8;

FIG. 12A is a side elevational view of the roller arm of FIG. 12;

FIG. 12B is a top elevational view of the transverse arm of the roller arm of FIG. 12

FIG. 12C is a longitudinal cross-sectional view of the transverse arm of the roller arm of FIG. 12B taken along line 12C-12C;

FIG. 13 is a perspective view of the handle body nozzle of the medical device of FIG. 8;

FIG. 13A is a front elevational view of the handle body nozzle of FIG. 13;

FIG. 13B is a side elevational view of the handle body nozzle of FIG. 13 having a set screw therein;

FIG. 13C is a further side elevational view of the handle body nozzle of FIG. 13 without a set screw therein;

FIG. 14 is a perspective view of the handle body of the medical device of FIG. 14;

FIG. 14A is a side elevational view of the handle body of FIG. 14;

FIG. 14B is a front elevational view of the handle body of FIG. 14;

FIG. 15 is a front perspective view of the handle body cap of the medical device of FIG. 8;

FIG. 15A is a rear perspective view of the handle body cap of FIG. 15;

FIG. 15B is a side elevational view of the handle body cap of FIG. 15;

FIG. 15C is a front elevational view of the handle body cap of FIG. 15;

FIG. 16 is a side elevational, broken view of an example heating rod for use with embodiments of the medical device as shown in FIG. 1, 2, 8 or 18-20 herein;

FIG. 17 is an example of a circuit diagram for the heating and control circuit for use in embodiments of the medical device as shown in FIG. 1, 2 8 or 18-20 herein;

FIG. 18 is a perspective view of an alternate embodiment of a roller head and roller arm combination for use with the medical devices herein;

FIG. 19 is a perspective view of a further alternate embodiment of a roller head and roller arm combination with an alternative nozzle head attached thereto; and

FIG. 20 is a perspective view of a yet a further alternate embodiment of a roller head and roller arm combination for attachment to an alternative nozzle head as shown in FIG. 19.

### DETAILED DESCRIPTION OF THE INVENTION

The invention herein is directed to a medical device for wound care sealing applications and a method of using such a medical device for wound care sealing. The method of using such a medical device can be used to secure, e.g., a barrier seal, barrier seal compound or other wound care compound, paste or adhesive to a patient.

As used herein, words such as “inner” and “outer,” “top” and “bottom,” “front” and “back,” “left” and “right,” “exterior” and “interior,” “up” and “down,” “distal” and “proximal” and words of similar import are used herein to aid in understanding the invention hereof with reference to the drawings and are not intended to be limiting to the scope of the invention.

The medical devices herein may be used with any medical wound care products that include an adhesive, paste or other cohesion compound alone or with an already applied barrier seal devices such as discs or wafers. While the medical devices may thus be used with a variety of medical appliances and wound care dressings for treatment of stoma, fistulae and other open wounds, the preferred embodiments will be described herein with respect to use of the medical devices with products known in the art as “barriers” or “barrier seals.” “Barriers” and “barrier seals” are intended to cover any wound care physical barrier product (generally referred to herein as a “barriers”) or its related adhesive or cohesive product or other sealing compound (“barrier compound”) and are used herein interchangeably to refer to the same group of products known generally and collectively as “barrier products.” Thus, unless specified otherwise, any reference to a barrier, barrier seal or other physical barrier product or barrier compound herein should not be viewed as limited and should be read broadly to physical seals, wafers, discs, covers, adhesives, cohesives, sealing compounds that are used in such wound care dressings or in installation of a related medical appliance for wound care, such as an ostomy pouch.

Reference to use of “barrier products” generally with the medical devices of the invention should also be interpreted to include but not be limited to a single barrier compound alone or used with a barrier (that may or may not be self-adherent), or to a combination of one or more barriers (that may or may not be self-adherent) layered or laminated together and/or in use with one or more layers of one or more types of barrier compounds.

An example of one such commercial barrier product that may be used with the medical devices herein includes a Cohesive® barrier seal and its associated barrier compound available from TG Eakin, Ltd, of Ireland. The Cohesive® barrier seals are intended to work with Cohesive® paste for filling in skin irregularities. Other similar products are known in the art. Such products are known and used by large numbers of ostomy and other wound care patients. Such wound care products can be used as part of many wound care dressing management programs, and in various different ways. When a barrier seal, such as Cohesive®, is applied peri-wound, and adhered to irregular surfaces and/or the highly individual body contours, use of the barrier compound (paste) acts as a foundation, thereby leveling and smoothing areas of contour or surface variances. The Cohesive® paste compound and similar products are primarily used to fill in crevices, and smooth uneven body surface features and contours around a stoma, fistula or wound.

The Cohesive® paste and other similar barrier compounds may also be used to enforce the structural support and adhesion around stoma or fistula appliances' wafers for added strength and security in a pouch or bag attachment or to reinforce an already applied product (or reattached product). Note that as an ostomy or other similar effluent pouch becomes full with effluent (liquid and/or solid), the weight thereof pulls on the support wafer, which can cause detachment and/or leaking or spilling. Thus, the paste compound is placed around and on top of the wafer, for added strength and added reinforcement. The paste compound may also be used by itself around a wound as a barrier, and can also be used on broken or compromised skin. Such barrier products, including barrier and barrier compounds, such as Cohesive® are extremely versatile, and are made more effective through use of the medical devices herein.

Such barrier products, including barriers and their compounds can be used in many areas of wound care, including securing a barrier seal by being moldable, absorbing moisture, allowing for a particular size barrier seal to fit varying sized wounds, keeping the skin barrier safe when applied to broken or compromised skin, or fixing a break in the skin or a skin barrier so as to rejoin the broken aspects, and may be used in one layer or in varying layers. Such barrier products further do not dry out and may be made to avoid allergy-inducing chemicals. As a result, in all of these areas, the medical devices herein also have applicability for installation of such barrier products.

In current practice, once a barrier product, such as a barrier and/or a barrier compound are molded and warmed per their manufacturer's guidelines, they are positioned onto a patient's skin by hand in a desired location, and then manually pushed into place using the tips of the fingers or other techniques noted in the Background section hereof

The medical devices herein improve such installation methods and provide hand-held medical devices configured for medical use that incorporate a roller head(s). Preferably such medical devices have one or more removable and interchangeable, roller head(s) for use in different end applications. The roller head(s) preferably further include protrusions which may be of varying shapes and sizes. The medical devices herein help to secure barrier products, such as barriers and barrier compounds in a highly effective manner by increasing surface area adherence, and increasing the contact points for cohesion between, e.g., a barrier seal, and/or any associated barrier seal compound or other adhesive, and a patient's skin surface.

The method of use of the medical device herein is preferably initiated by rubbing or rolling one of the preferably interchangeable roller heads over a barrier product, such as a barrier and/or its barrier compound, while applying an indicated and/or appropriate level of pressure, which level of pressure may be varied depending on the thickness, specific gravity and/or viscosity of the barrier and/or its associated barrier compound or the quantity of barrier compound applied (i.e., the mass or layer thickness as measured transversely across the layer extending outwardly from the outer surface of the patient's skin).

The novel medical devices herein and their method of use provide a significant and substantial increase in adhesion, which in turn contributes to a decrease in leakage, spilling and/or detachment of a wound care appliance or of a dressing applied to a patient having a stoma, fistula or open skin wound over that achieved using the prior art standard practice of manual application. It further provides beneficial and more uniform adhesion without the use of currently and commonly used more risky techniques such as use of scissors, tongue blades and/or hemostats, hair dryers and similar implements and devices. It also provides a significant reduction in labor time involved in manually attempting to form indentations with a single tipped pair of scissors, a tongue blade and/or hemostats or in using a hair dryer.

Many of the current, commercially available compounds and barrier seals available are typically activatable through a body's surface heat (and/or the applicant's hands) or an external heat source during application, so as to provide softening and moldable pliability to an otherwise somewhat rigid material or compound. The medical devices herein are optionally provided with an additional benefit for reducing dressing time by providing a faster, more efficient and effective way of applying an adhesive while also heating the roller head.

The medical devices herein and their method of use increase adhesion properties by increasing and making more uniform the points of substrate contact (i.e. barrier seal compound to a patient's skin) and also by increasing the surface area of adhesion, thereby increasing medical dressing productivity, containing costs, decreasing dressing labor effort and time, decreasing the risk of infection, and improving quality, integrity and longevity of wound dressings in which barrier and associated barrier compounds are used as a dressing management program for any type of indicated stoma, fistulae, or other wound or opening of the body requiring a medical dressing.

The medical devices herein can be used when applying various skin barriers and associated barrier compounds as noted above, including those that contain hydrocolloid or other similar compounds as part of stoma, fistula or other wound care dressing during initial placement, during a dressing change, in enforcing placement, or when reinforcing, re-sealing or at any other stage in dressing and/or wound care processes. The devices assist, and work by enforcing, re-enforcing, and/or securing barrier(s) and/or associated compound(s) by creating an even and uniform application on a patient's skin. In preferred embodiments, this effect is enhanced by the devices' a roller head(s) that create(s) micro-indentations by use of protrusions on an exterior surface of the roller head, which protrusions may have varying shapes.

In some embodiments, the protrusions may have a rivet-shape or may simply appear as blunt head protrusions. Such protrusions impress micro-indentations of a relatively corresponding shape into the compound or adhesive, thereby allowing for an overall increase in adhesion properties of the hydrocolloid, or other barrier seal compounds or adhesives to surfaces of substrates, especially when the skin surface has irregularities.

The method of use of the medical device herein secures, applies, enforces, and/or re-enforces barriers and/or barrier compounds to the skin in any wound dressing regimen involving a stoma, a fistula or other open wound, including those in which an effluent-producing wound dressing is required, such as those used with ostomy pouches and similar applications, which wounds arise either surgically or non-surgically, wherein such wounds require use of the above-identified types of barriers and barrier compounds.

The medical devices herein, are hand-held medical devices that preferably include a handle, which may be of a varied size and shape. The handle supports the rolling head, and such rolling head can be made to be interchangeable with one or more rolling heads having varying sized blunt or round protrusions, or a more rivet-shaped protrusion. The rolling head(s) are preferably manually rotatable. The protrusions may be applied in varying patterns so that they are arranged in rows, and are either aligned or staggered.

The rolling head(s) may be fixed or releasably attached to a roller arm through one or more suitable fastener mechanisms. Alternatively, the rolling head(s) may be formed so as to include or be composed of one or more fixed or releasably attached independent rolling wheels having on an outer surface thereof a series of protrusions. In both the fixed or releasably attached embodiments of the rolling head(s), the outer surfaces (or outer surfaces of various independently mounted wheels) can be made to be interchangeable so as to provide varying application effects.

The medical devices herein are indicated for personal or professional use and can be made in varying sizes so as to be useful in different physical body locations, and for different surface areas and wounds to be treated. The device may be sized and configured to suit highly individualized wound dressing needs of patients.

When barrier compounds (i.e., pastes, powders or other adhesives and cohesives) are used as part of a stoma dressing regimen, the patient's preferred barrier compound and any associated barrier is placed manually, i.e., by hand, around the border or edges of the wound (peri-wound). Because the anatomy of the human body's surfaces contain folds, wrinkles, irregularities, deformities or scars, and other protrusions or indentations, when the barrier and its associated compound, e.g., the Cohesive® products are applied peri-wound, and adhered to the irregular surfaces of the highly individual body contours, the barrier compound acts as a ‘foundation’—leveling and smoothing areas of contour or surface variances.

The barrier and/or its associated barrier compound for the same purpose and use, is strained to bring the compound into contact with the irregular contours of the body's surface. Once the barrier and/or its associated barrier compound of choice is placed, it can then be secured using the medical devices herein.

The medical devices herein are placed onto the barrier compound after the barrier compound has been placed onto the skin. The operator holds the handle, and rolls the medical device over the barrier compound with sufficient pressure. The medical devices may be rolled either in a “one way” direction (i.e., across a surface and then lifted, placed back at the starting point or another point and similarly rolled in one direction across the surface), in a “back and forth” manner (rolling the device across the surface in one direction and then back in another direction either at the same or a different angle), or in a “circular” manner (i.e., around the periphery or using circular motions across the surface. In addition the roller may be used to roll and press or indent into the surface. The rolling head can thus be employed in various motions as may be considered to be preferable to a particular patient, and that patient's variable wound shape and characteristics.

This rolling head protrusions form “rivet” shapes or other curved or blunt “micro-indentations” into the barrier compound. Introducing such “micro-indentations” into the barrier compound thereby increases the surface area into which the two substrates (the barrier compound and the skin) may be adhered. As a result, there is increased adhesion of the barrier compound to the skin (whether it is a separate layer, the backing on a barrier product or part of a laminated structure), due to the more adherent substrate contact points created through use of the medical devices herein. This creates a very beneficial, substantial increase in adhesion, which in turn, can provide associated and significant decreases in leakage of body effluent.

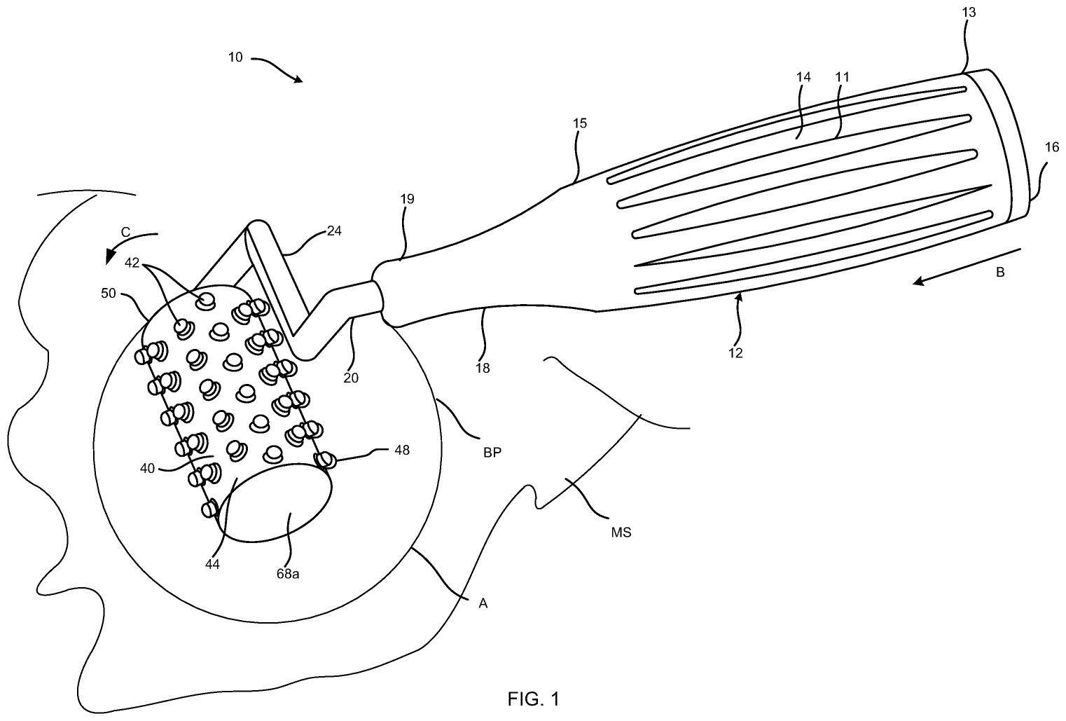

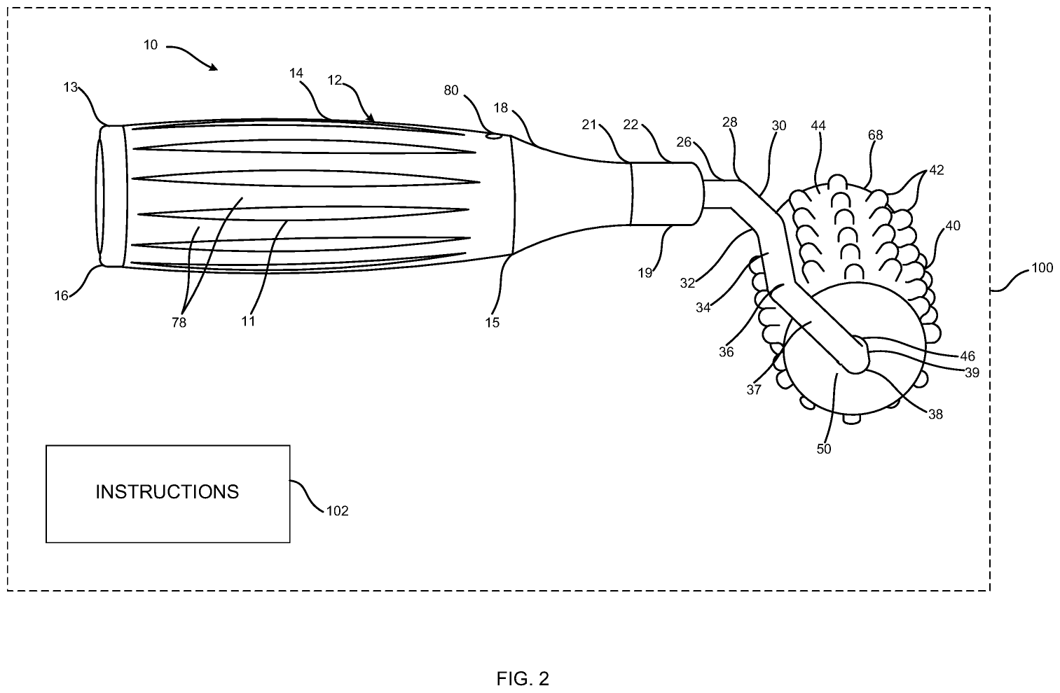

The invention will now be described in preferred embodiments herein with reference to the attached drawings. In the embodiments of FIG. 1 and FIG. 2, the devices are operationally the same, but in FIG. 1 the handle body, handle body nozzle and roller arm connector are formed as a single unitary body and in FIG. 2, these components are shown as releasably detachable components. As other aspects of the device are similar, analogous reference numbers are used to show corresponding areas of the device. Analogous reference numbers are used herein for corresponding components in alternative embodiments. With reference particularly to FIGS. 1 and 2, the medical device is referred to generally herein as medical device 10.

As best shown in FIG. 2, the medical device 10 of the embodiment shown includes in principle parts a handle 12, a roller arm 20 and a roller head 40.

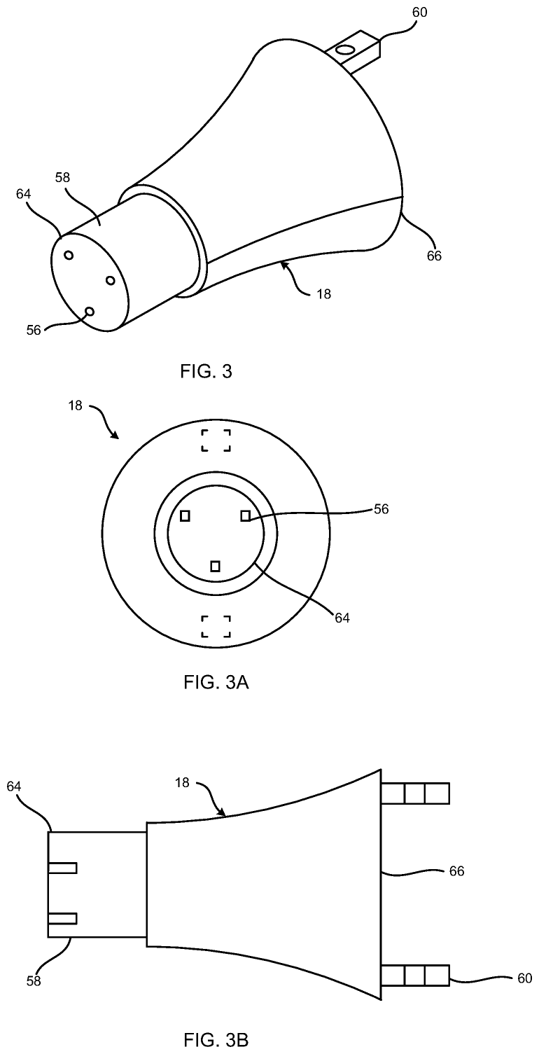

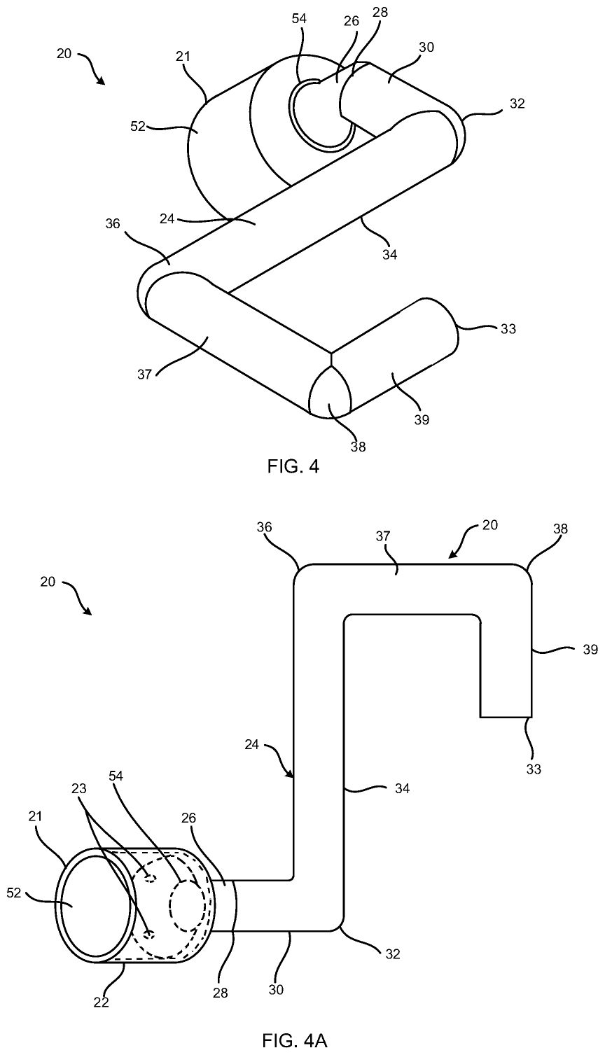

With reference to the handle 12, the handle and its constituent parts are shown in FIGS. 1, 2, 3-3B, 5-5B and 7-7B as including a handle body 14, a handle body nozzle 18 and a preferred handle body cap 16.

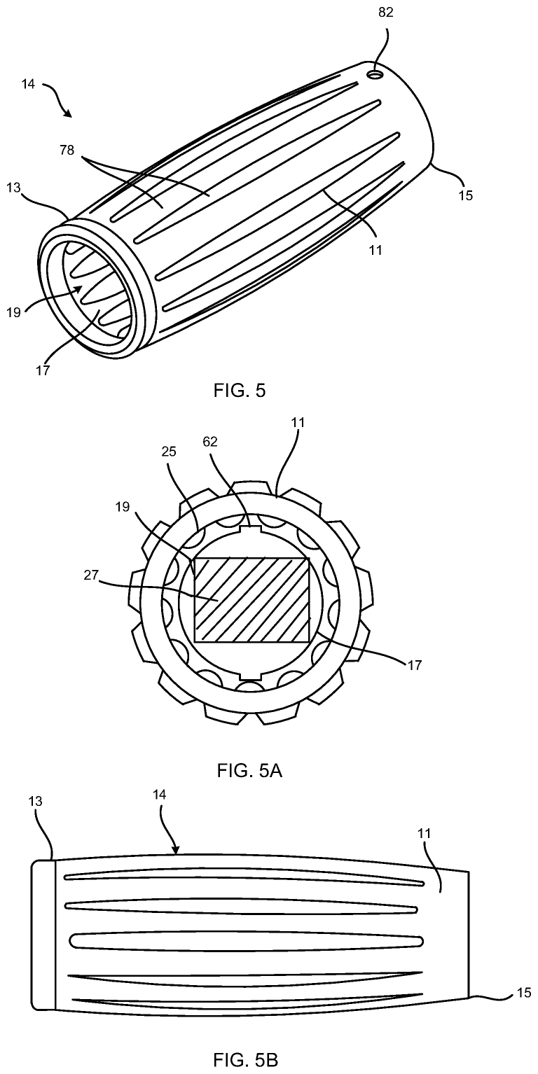

As shown, the handle body 14 is made to be relatively slim-handled in width for ease and comfort of the user when manually using the medical device 10. However, it is within the scope of the invention to vary both the length as measured longitudinally along the handle body 14 and the width of handle body as measured transversely across the handle body 14. The handle body 14 is also shown as having a round cross-sectional shape (as best shown in FIG. 5A), however, the shape may be varied to be square, triangular, oval, elliptical and the like in cross-section. For ergodynamic purposes, a gel outer layer could be applied if desired for soft gripping. As shown, the outer surface has features such as ribs 78. Such features are to assist in gripping, although any outer surface configured to facilitate gripping may be used if such feature is desired. The handle body 14 is preferably about 2 in. to about 6 in. in length, preferably about 3 in. to about 5 in. in length, and about 0.25 in. to about 1.5 in. in width, preferably about 0.5 to about 1.25 in. in width. The overall profile may be straight along the length or slightly curved along its length as it approaches the distal end 15 of the handle body 14.

As shown in FIG. 1, the handle 12 and the handle body 14 may be made to be as a unitary one-piece handle body with fixed proximal and distal ends that are not releasably detachable as can be done for easy-to-use disposable devices, devices without electronics and for various lightweight or inexpensive kits, e.g., for home use or sterile hospital use. It is also possible to combine aspects of the device 10 as shown in FIGS. 1 and 2 to have an optional closed end, e.g., the proximal end of the handle body, but have a releasably detachable distal end where the handle body 14 meets the handle body nozzle 18.

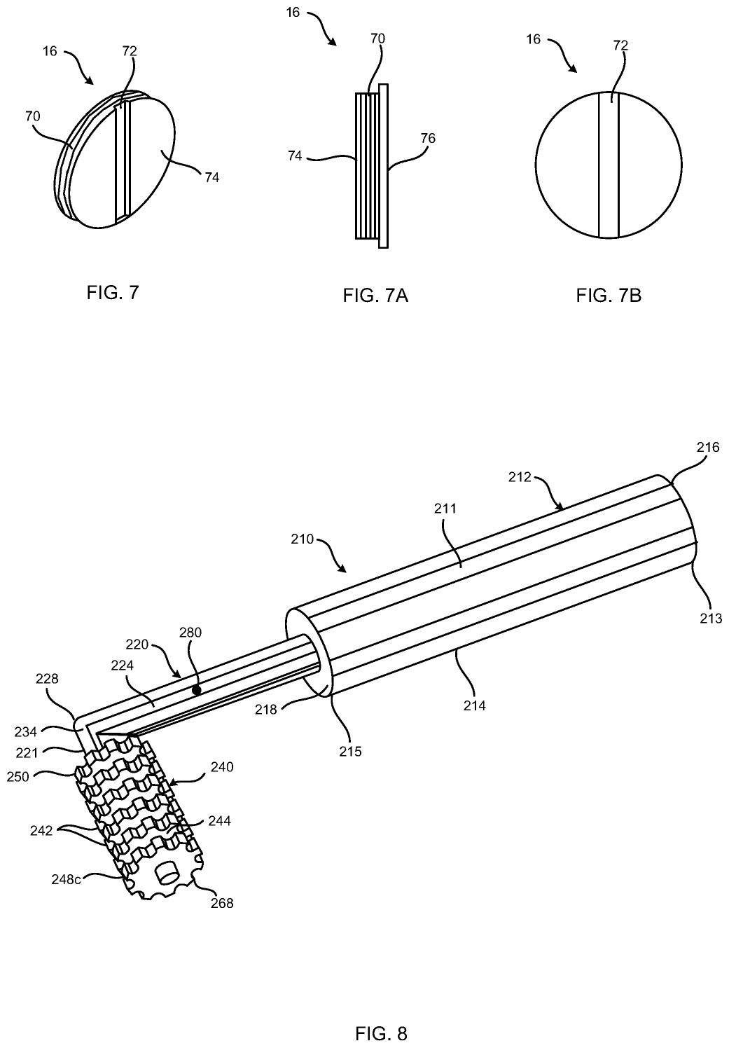

As shown in FIG. 2, the handle body 14 includes open ends on both sides that are detachably connected to an end cap 16 on a proximal end 13 of the handle body 12. The cap 16 as shown in FIGS. 7-7B can be of varied design. In the embodiment shown, the end cap 16 may have side edges 70 that include screw threads or other features that can be made to interact with mating interior features on the proximal end 13 of the handle body 12 on its interior surface 17. The end cap 16 may also include a groove or channel 72 on an exterior side 74 of the cap for engaging a flat headed tool for removal or tightening of the end cap. However, it should be understood that an end cap 16 may be welded, glued, snap fit, connected by fasteners or otherwise attached (or made to be releasably detachable) to the proximal end 13 of the handle body. The cap is preferably sized to fit smoothly and attach or be formed with the proximal end 13 of the handle and so may vary in size, shape and width depending on the handle body width and desired thickness. Preferably the end cap is smaller in width than the outer width of the handle body noted above so as to fit within the interior surface 17 of the handle body.

The handle regardless of configuration should preferably be useful and capable of serving as a handle for manual use by either a medical professional or a patient as the end-users. The handle body and end cap may be made to include metals such as titanium, aluminum and the like, metal alloys such as stainless steel, aluminum allows or the like, polymeric and composite materials that are preferably heat moldable, whether reinforced or non-reinforced, or ceramics, such as polyarylenes, polysilicones, polyacrylonitriles, polyacrylonitrile butadiene styrenes, polyamides, polyimides, polyamideimides, polyacrylamides, polyvinyl chlorides, polytetrafluoroethylene, moldable fluoropolymers, polyurethanes, polyureas, and the like, as well as co-polymers, blends and alloys of such polymer and composite materials, or moldable or sinterable ceramics. Such materials should be suitable for the external medical uses intended, including in a wound care area and/or in preferred hospital uses and/or in conditions that require sterilization, and for preferred embodiments in which heating is also employed, are heat resistant and fire-resistant. Combinations of such materials may also be used. Suitable materials are preferably also able to withstand the level of force and pressure needed to sufficiently employ the roller head to carry out the purpose of the invention as described herein. The handle body may be made to be available for disposable one-time use, for limited uses, or for long-term use in which it is easily cleaned, reuseable and/or sterilizable. Further, it may be made to be applicable for both personal, and or professional use.

In use, the handle 12 and handle body 14 are intended for use manually and to be held by a user's hand, finger, wrists, or otherwise affixed onto another stationary object so that the device can be used without use of the hands, arms, or fingers in certain circumstances; or can be otherwise constructed or designed that the patient (even if handicapped) or any other person such as a medical professional can operate the medical device 10.

The handle body 14 may be formed as a solid piece (whether unitary or non-unitary with an end cap and/or a handle body nozzle) or can be formed so as to have a hollow interior space 17 defined by an interior surface 17 of the handle body 14. It is also optional and possible to provide an internal and optionally removable sleeve 25 into the interior space 17 of the handle body 14. Such a sleeve can be used to shield and/or protect optional internal components and can be made to facilitate removal of such internal components for maintenance, repair or replacement.

In one embodiment, internal electronic components 27 and/or wiring can be provided. Such internal electronics can be any of those known in the art or to be developed that can be employed for electrical control of, e.g., the roller head (which may be manual or electrically driven such as by an internal small gear or other minor limited motor), or to power a heating element. A heating element or other heating element may be provided within the handle body 14 to heat the medical device 10 through wiring or conductive heating which is preferably variable in heat level so that if heat is desired in the use of the device for heating the barrier product, it can be easily delivered. A heating element may be employed as one of the optional electronic components 27 either within an interior space 19 of the handle body 14 or even optionally in the roller arm or roller head. If variability in heating is desired, a heat adjustment toggle may be employed with a limiting device for heating the heating element to variable levels as desired.

Such electronic components and/or wiring (heating elements, motors, power sources such as batteries, lighting sources and/or electrical connections) can be adopted in unitary one-piece handle designs and/or in the associated roller head of such a device, in a handle body having only one open end (such as a device with an end cap and unitary handle body nozzle) or in a handle body as shown in FIG. 2 wherein the handle body 14 has an end cap 16, and a handle body nozzle 18 that are all releasably detachable and located in the handle body or inside of a hollow roller head or in the roller arm. Regardless of location, any optional heating element should heat the device 10 only to a skin safe temperature to avoid harm or burning of a patient or user.

The handle body nozzle 18 if a separate and detachable component may be attached to the handle body 14 at the distal end 15 thereof by various mechanisms. As shown, the proximal end 66 of the handle body nozzle 18 is fitted with snap-fit protrusions 60 that can fit within mating grooves 62 in the interior surface 17 of the handle body 14. Other snap-fit, friction fit, mating features, adhesives or welds may be used for this purpose. As shown, a set screw 80 may be used to securely fasten the handle body 14 and handle body nozzle 18, wherein the set screw passes through opening 82 defined in the handle body 14 extending inwardly through the exterior surface 11 thereof. The proximal end 66 of the handle body nozzle 18 is also preferably of the same or a similar width and shape for a smooth transition between the handle body 14 and the handle body nozzle 18, although a rim or edge for facilitating removal is within the scope of the invention or any enlarged design feature (shield, curve or shape provided for safety or aesthetic purposes). The distal end 64 of the handle body nozzle 18 preferably has a distal portion 58 that is configured and shaped to fit within a roller arm attachment 22. As shown, the roller arm attachment is a roller arm connector attachment 22 (referred to herein also as a roller arm connector for shorthand reference purposes).

In the embodiment shown, if electronic components are provided, the handle body nozzle 18 can be configured as shown to have within the distal portion 58 an electrical connector 56. Such connector can be of a variety of types of suitable electrical connectors as are known in the art, or to be developed, and, preferably interact with mating connectors provides electrical connection from a power supply (such as a battery or electrical wiring extending through the device to communicate with an electrical plug) to any electrical components within the handle body 14, roller head 40 or roller arm 20. As shown, the roller arm connector 22 may have mating connection features such as receiving connector holes 23 on a first connector side 52 of the connector 52. As such the connector holes 23 may serve as a female connector portion within the connector 22 that can mate with a male connector 56. However, it should be apparent that other connectors may be used or their positions reversed if desired.

When the handle body nozzle 18 connector is securely fastened to the roller arm connector 22, it provides a secure electrical connection for the roller arm 20 and rolling head 40, which can also enable interchangeability of the roller head 40 alone or in combination with a roller arm 20 so that the entire distal combined portion of the device having the roller arm and roller head may be interchangeable if desired.

The roller arm 20 connector 22 is preferably an interchangeable attachment that may be releasably secured and when connected is both securely and preferably hermetically sealed against the distal end portion 58 of the handle body nozzle 18 and is connected also on a mount end 33 to the roller head 40. If electronic components are provided, a power supply preferably within the interior space of the handle body provides an electrical source and connection into the roller arm 20 and rolling head 40 components which are preferably each interchangeable alone or independently.

The roller arm 20 preferably includes an angled roller arm body 24 extending from a second side 54 of the connector 22 on the connector end 21 of the roller arm 20 to a mounting end 33. The roller arm body 24 may be designed in various shapes and configurations to suit aesthetics and/or utilitarian operation but is preferably configured to extend from the second side 54 of the connector 22 at the connector end 21 and bend or curve around the roller head 40 so that the mounting end 33 fits within an opening 46 defined by the a mounting side 50 of the roller head 40. The opening may be a snap fit hole that engages the mounting end 33, a friction fit, a rotatable cam or similar mechanical roller arm mountings. Such rotatable mountings are known in the art and may be modified as desired. It is preferred that the roller arm is formed of a material such as those used for the handle body and connector end 21 of the roller arm to allow for heating and/or electrical operation if desired. The roller head 40 may be solid with a portion that defines the receiving opening for the roller arm or hollow for incorporating desired electronic components as noted above.

In a preferred embodiment as shown herein, the roller arm body 24 preferably includes the connector 22 as noted above that is configured to mate with the distal end portion 58 of the handle body nozzle 18 on a first side 52 of the connector 22 and to engage on the opposite side 54 of the connector 22 to a first longitudinally extending portion 26 running from the connector 22 to a first bend 28 to a more downwardly extending first angled portion 30. The first angled portion 30 then undergoes a second bend 32 into a first transversely extending portion 34 that runs from the bend 32 in a direction parallel to but spaced from the exterior surface 44 of the roller head 40 until the first transversely extending portion 34 passes the end of the roller head 40 past the mounting side 50 of the roller head 40, at which point the roller arm body 24 again has a third bend 36 into a second angled portion 37. The second angled portion 37 runs to a fourth bend 38 extending between the second angled portion and a mounting portion 39. The mounting portion 39 extends transversely back towards the roller head 40 from the fourth bend 38 and into the mounting opening 46 in the mounting side 50 of the roller head wherein the roller arm body 35 terminates at a mounting end 33 of the roller arm 20.

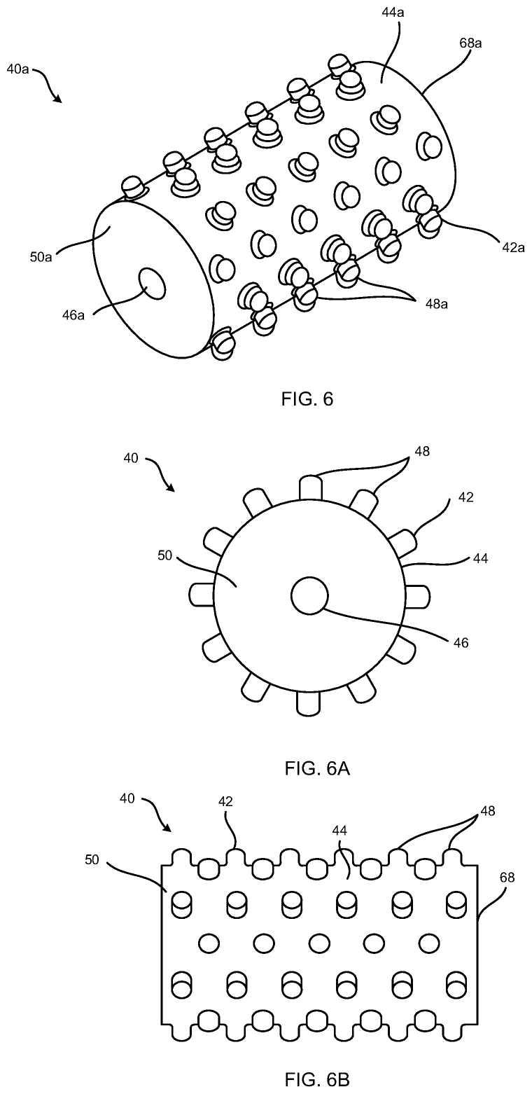

The rolling head 40 may be formed as a single unitary roller head body that is hollow (as noted above) or solid, either of which defines a mounting opening 46 for receiving at least a part of the mounting portion 39 of the roller arm body 24. The rolling head may also be formed as a series of parallel roller sections that appear as a “wheel”, as described below in embodiment 210 sitting side-by-side but spaced apart at regular intervals. Such rows may independently roll on an axle formed by an extending mounting portion or separate axle. In the preferred embodiment as shown, the roller has an exterior surface 44 that includes one or more protrusions 42. The protrusions as noted above may have various shapes and profiles but are preferably blunt in that they are not sharp. As shown in FIGS. 6A and 6B, blunt protrusions 48 are shown with a curved external end. In an alternative roller head 40a shown in FIG. 6 that may be interchangeable with the roller head 40, the protrusions 42a are also blunt but have a rivet-like shape 48a that has a blunt taper at the end thereof Whether a curved end or “blunt” end or a rivet-like shape, such protrusions are preferably not sharp so as to avoid hurting the patient and are preferably blunter so as to perform the function of increasing surface adhesion between a barrier product and mammal skin in an area near a wound.

The roller head 40 is preferably attached to the mount portion 39 of the roller arm body 24 by friction fit, mounting washers or use of a screw on either side of rolling head cylinder depending on how far the mounting portion of the roller arm extends through the roller head. As shown, it extends only partially into one side 50 of the roller head, but if preferred could continue so that the end 33 of the mounting portion 39 extends outwardly (not shown) through the opposite side 68 of the roller head 40. It is also possible to have the roller arm body fork outwardly from the bend 32 to allow a transverse extension portion to go in two opposite directions and to connect by angled portions on either side of the roller head so as to have two mounting end portions.

The protrusions 42, 42a of the interchangeable roller heads may be offered in a variety of sizes (and shapes) to meet specific patient needs and to suit the particular barrier product being employed. For example, a “flat” tip protrusion 42 could be employed in one embodiment of an interchangeable roller head so as to allow for a closer seal proximity between an actual wound border or wound edge and the barrier compound or other barrier product being applied.

The roller head and its protrusions may be formed of the same materials noted above, including surgical stainless steel, aluminum, titanium and other metals and their alloys, polymer and composite materials, composites and other suitable materials as note above. The protrusions herein can gently assist in successfully obtaining a leak-proof mold and seal around the actual stoma or other wound area by pressing the innermost edge of the barrier compound seal, or the collar thereof circumferentially until a full rotation is made around the edge of the wound and the barrier compound is indented or pressed by flat tip head, securing and increasing the border edge integrity between the compound and the wound.

The medical device 10 may be provided alone or in a kit 100 as shown schematically in FIG. 2 that includes one or more of the medical device a described above in detail, having a handle such as handle 12, a roller arm, such as roller arm 20 and a roller head 40 having an exterior surface 44 on the roller head 40 configured to enhance adhesion of a barrier product such as BP in FIG. 1 to an area A of mammal skin MS near a wound opening. The kit 100 preferably also includes instructions 102 describing how to use the medical device 10. If the medical device 10 is formed so as to have interchangeable roller heads (or roller head and roller arm combinations), one or more additional such interchangeable roller heads and/or roller arms may be provided to the kit. A barrier product BP as described herein may also be incorporated into the kit 100 such as the barrier products described hereinabove. Such barrier products may include a barrier and a barrier compound as described also above. A carrier case or other suitable container may be provided to the kit as an optional feature (not shown).

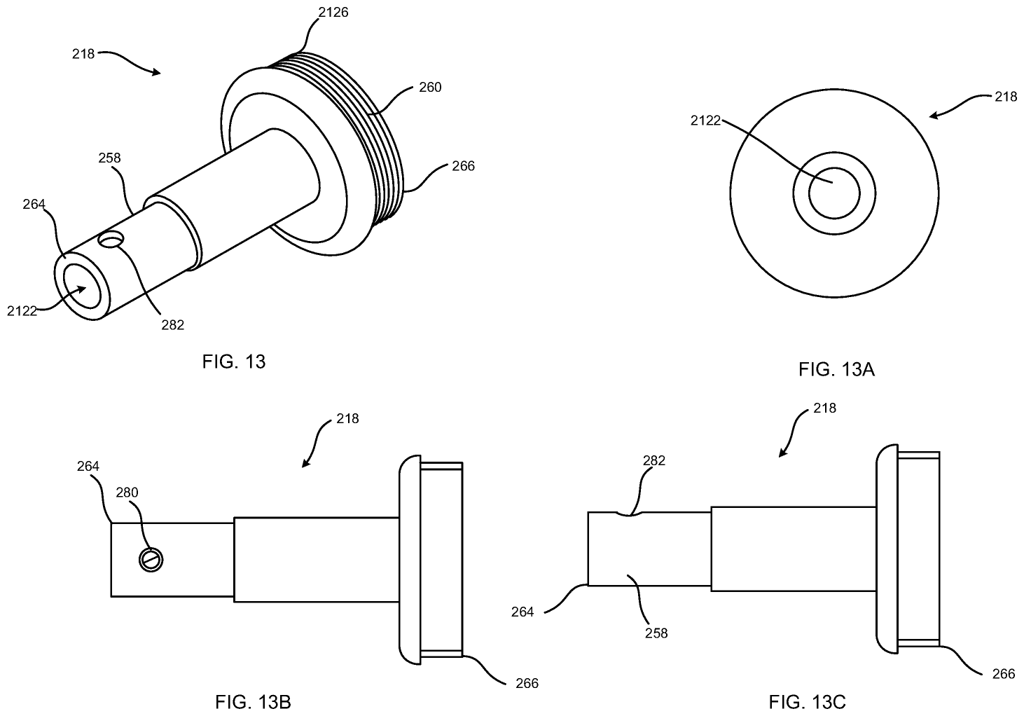

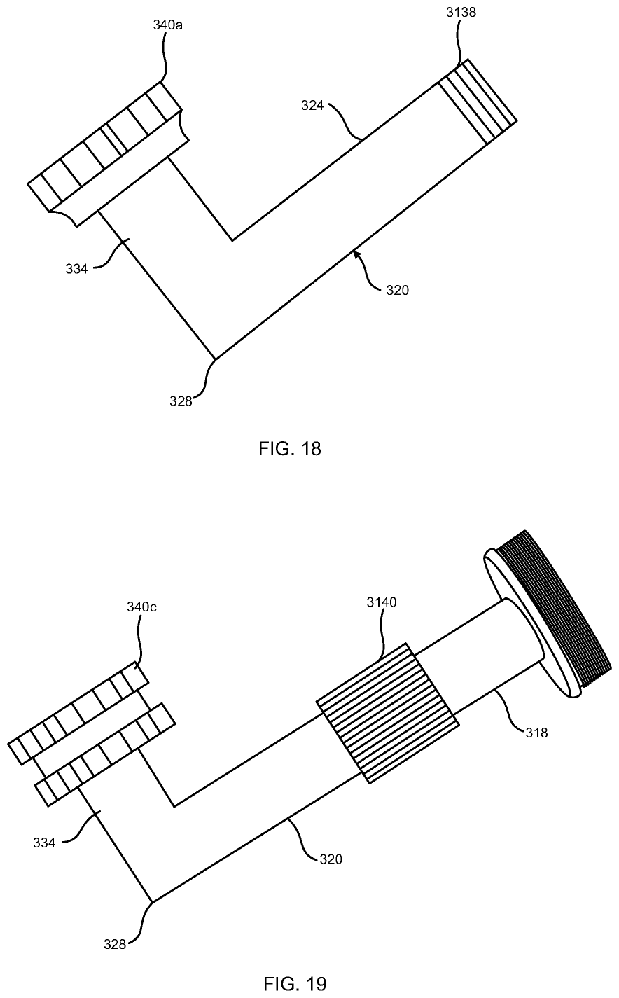

Further embodiments of the invention are shown in FIGS. 8-15 as embodiment 210 as well as in FIG. 18-20 as embodiment 300. The primary portions of the devices are operationally similar to that of FIG. 2, having a handle body, a handle body nozzle, a roller arm connector and roller head. As various aspects of the medical devices which are similar and analogous have analogous reference numbers to show corresponding areas of the device where applicable. With reference particularly to FIGS. 8-15, the medical device is referred to generally herein as medical device 200.

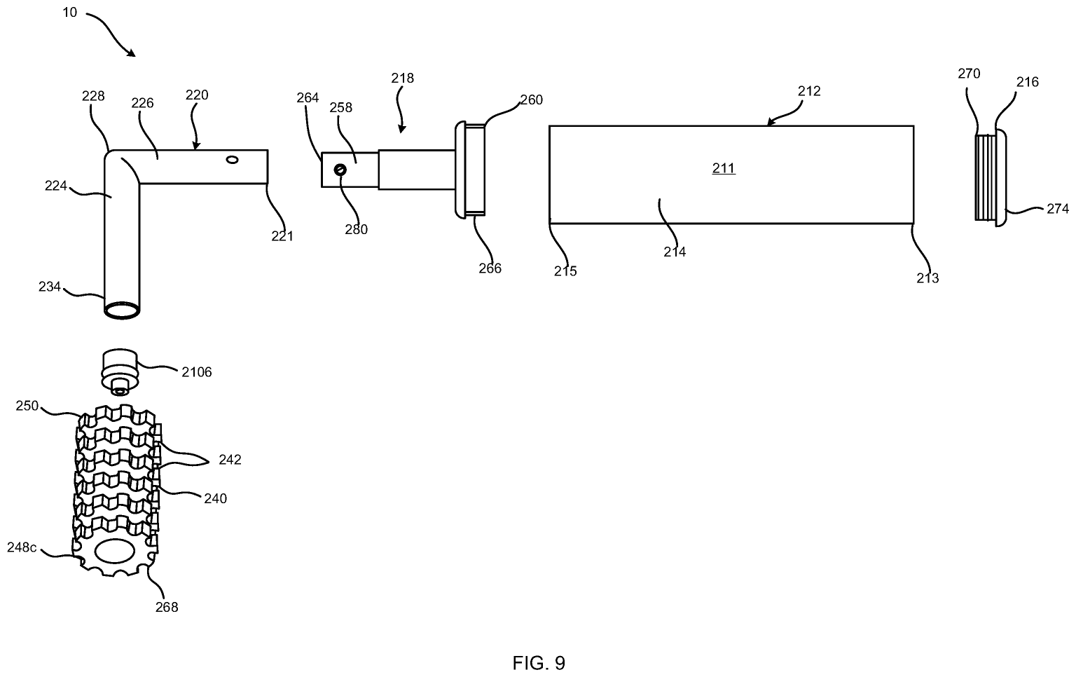

As best shown in FIG. 9, the medical device 210 of the embodiment shown includes in principle parts a handle 212, a roller arm 220 and a roller head 240.

With reference to the handle 212, the handle and its constituent parts are shown in FIGS. 8, 9, 13-13C, 14-14B, 15-15C as including a handle body 214, a handle body nozzle 218 and a preferred handle body cap 216.

As shown, the handle body 214 is made to be relatively slim-handled in width for ease and comfort of the user when manually using the medical device 210 in a manner similar to that of embodiment 10. However, it is within the scope of the invention to vary both the length as measured longitudinally along the handle body 214 and the width of handle body as measured transversely across the handle body 214. The handle body 14 is also shown as having a round cross-sectional shape (as best shown in FIG. 8), however, the shape may be varied to be square, triangular, oval, elliptical and the like in cross-section. A gripping surface may be provided and the handle modified as discussed above. The handle and its nozzle in such embodiment may be formed of the materials as noted above, and preferably are formed of a medical grade stainless steel, such as Type 316 or Type 440 stainless steel. The handle and end cap, described below, are preferably each 440 stainless steel, and the handle and roller arm may be preferably 316 stainless steel. The handle may be hollow as shown or a solid rod. If hollow, it is preferably milled from a single bar. The handle may also include an electrical connection incorporated in the handle nozzle in a manner as shown and described in the embodiment 10. As shown in the drawings, for convenience purposes, the handle and nozzle are hollow, but each may be formed as a solid tube also having an optional sleeve in the handle interior as in embodiment 10 internally, and/or each having electrical components and/or heating elements and controls therein. Such components may be formed of suitable aromatic polymers and plastics such as polyarylenes, polyphenylenes, polysufones, and the like or other suitable component polymers such as polyimides, polyamides and polyolefins if acceptable for electronic grade usage.

The handle body 212 preferably may be sized as noted above in embodiment 10 and having an overall profile that is straight along its length as it approaches its distal end 215 of the handle body 212.

The handle body 212 may be made to be as a unitary one-piece handle body with fixed proximal and distal ends that are not releasably detachable in the same manner as embodiment 10, for easy-to-use disposable devices, devices without electronics and for various lightweight or inexpensive kits, e.g., for home use or sterile hospital use. It is also possible to combine aspects of the device 210 as shown in FIG. 8 for a closed end, e.g., the proximal end of the handle body, but have a releasably detachable distal end where the handle body 212 meets the handle body nozzle 218. The closed end as shown, is closed using an end cap 216.

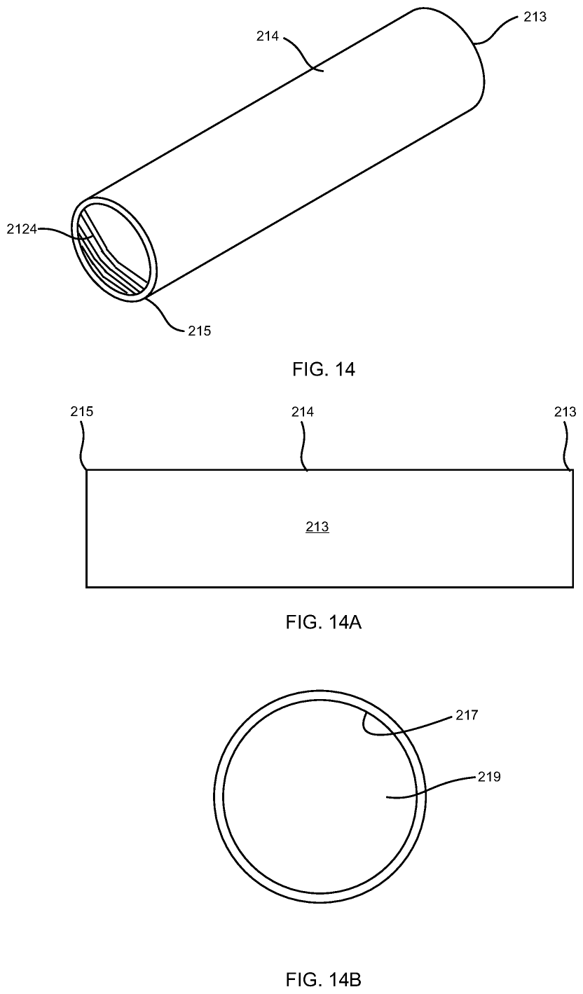

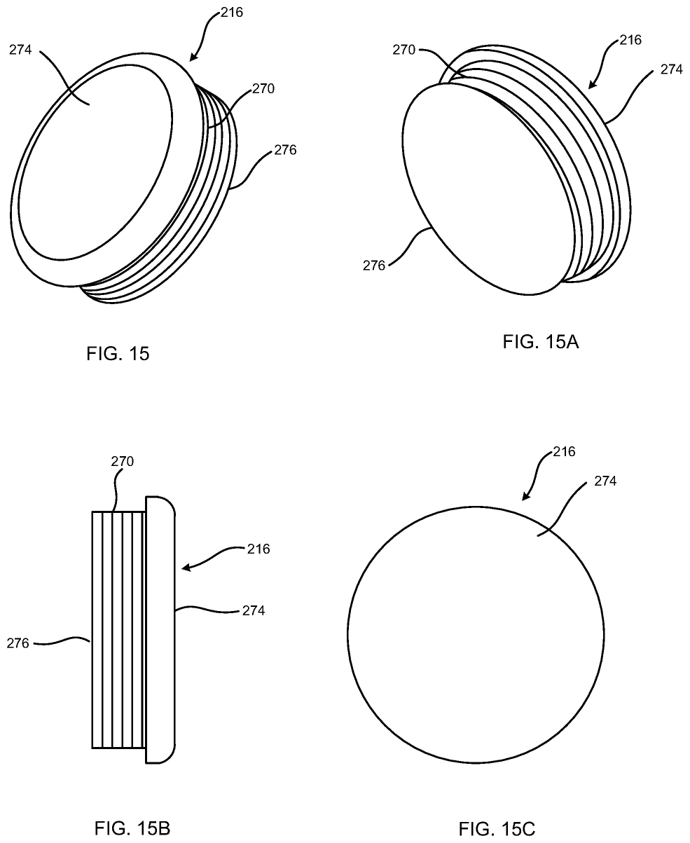

As shown in FIGS. 8 and 14-14B, the handle body 214 includes open ends on both sides that are detachably connected to an end cap 216 on a proximal end 213 of the handle body 214. The cap 216 as shown in FIGS. 1 and 15-15C can be of varied design. In the embodiment shown, the end cap 216 may have side edges 270 that include screw threads or other features that can be made to interact with mating interior features on the proximal end 213 of the handle body 214 on its interior surface 217. The end cap 216 has an exterior side 274 of the cap for removal or tightening of the end cap. The end cap 216 may also be hollow in its interior to hold a battery or other power source. While shown attached by screw threads, as with the embodiment 10, it should be understood that an end cap 216 may be welded, glued, snap fit, connected by fasteners or otherwise attached (or made to be releasably detachable) to the proximal end 213 of the handle body 214. The cap is preferably sized to fit smoothly and attach or be formed with the proximal end 213 of the handle and so may vary in size, shape and width depending on the handle body width and desired thickness. Preferably the end cap is smaller in width than the outer width of the handle body noted above so as to fit within the interior surface 217 of the handle body.

The handle regardless of configuration should preferably be useful and capable of serving as a handle for manual use by either a medical professional or a patient as the end-users. The handle body and end cap may both be made of similar materials as with the handles and end caps as noted above. Such materials should be suitable for the external medical uses intended, including in a wound care area and/or in preferred hospital uses and/or in conditions that require sterilization, and for preferred embodiments in which heating is also employed, are heat resistant and fire-resistant. Combinations of such materials may also be used. Suitable materials are preferably also able to withstand the level of force and pressure needed to sufficiently employ the roller head to carry out the purpose of the invention as described herein. The handle body may be made to be available for disposable one-time use, for limited uses, or for long-term use in which it is easily cleaned, reuseable and/or sterilizable. Further, it may be made to be applicable for both personal, and or professional use.

In use, the handle 212 and handle body 214 are intended for use manually and to be held by a user's hand, finger, wrists, or otherwise affixed onto another stationary object so that the device can be used without use of the hands, arms, or fingers in certain circumstances; or can be otherwise constructed or designed that the patient (even if handicapped) or any other person such as a medical professional can operate the medical device 210.

As shown herein, the handle is hollow. It is also optional and possible to provide an internal and optionally removable sleeve (see sleeve 25 in embodiment 10) into the interior space 217 of the handle body 214. Such a sleeve can be used to shield and/or protect optional internal components and can be made to facilitate removal of such internal components for maintenance, repair or replacement.

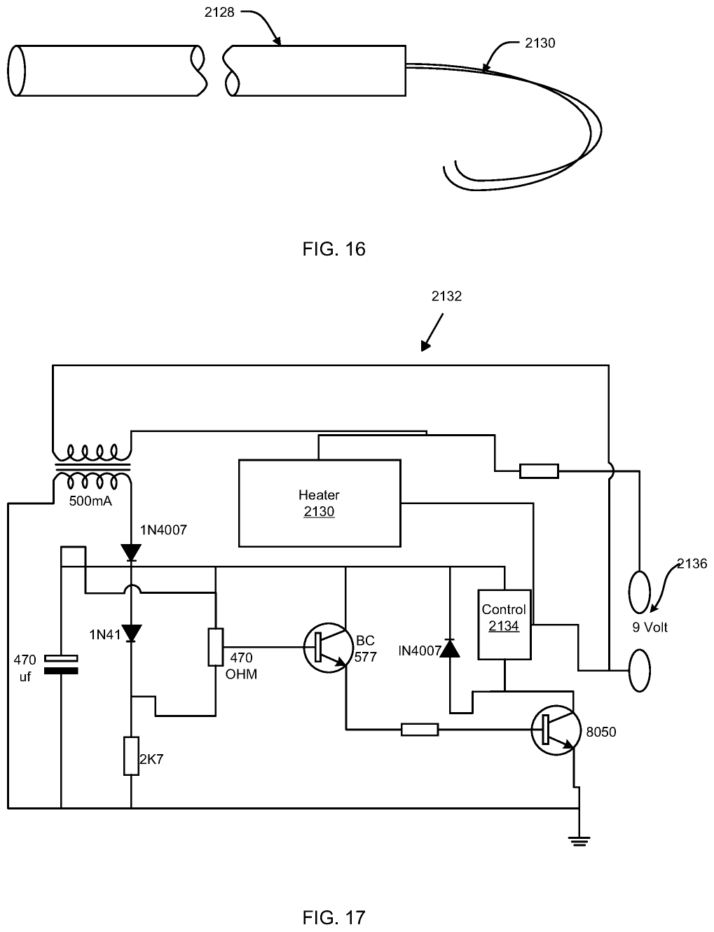

In one embodiment, internal electronic components (see components 27 in embodiment 10) and/or wiring can be provided. Such internal electronics can be any of those known in the art or to be developed that can be employed for electrical control of, e.g., the roller head (which may be manual or electrically driven such as by an internal small gear or other minor limited motor), or to power a heating element. A heating element or other heating element may be provided within the handle body 214 to heat the medical device 210 through wiring or conductive heating which is preferably variable in heat level so that if heat is desired in the use of the device for heating the barrier product, it can be easily delivered. A heating element may be employed as one of the optional electronic components 27 either within an interior space 219 of the handle body 214 or even optionally in the roller arm or roller head. If variability in heating is desired, a heat adjustment toggle may be employed with a limiting device for heating the heating element to variable levels as desired. As shown in FIG. 16, a rod heating element may be used in either embodiment 10 or 210. The heating element is a rod heating element 2128 having lead wires 2130 extending therefrom to be incorporated into an attachable electrical plug or into a chargeable battery power source. Suitable such rod heaters are commercially available as cartridge heaters, such as those available as a 10 W 1.1 Amp, 9 V DC or similar part (30 W; 3.3 Amp current) may be used. Lead wires are preferably heat resistant and coated such as with polytetrafluoroethylene (PTFE).

Such electronic components and/or wiring (heating elements, motors, power sources such as batteries, lighting sources and/or electrical connections) can be adopted in unitary one-piece handle designs and/or in the associated roller arm and/or roller head of the device. In the device as shown, the heating element may be in the handle, and other electronic controls and electronic connectors may be positioned in the handle nozzle (as described above) or in the roller arm and roller end cap as described below. Regardless of location, any optional heating element should heat the device 10 only to a skin safe temperature to avoid harm or burning of a patient or user.

The handle body nozzle 218 if a separate and detachable component as shown may be attached to the handle body 214 at the distal end 215 thereof by various mechanisms. As shown, the proximal end 266 of the handle body nozzle 218 is fitted with screw threads 2126 on proximal end 260 that can engage mating threads 2124 in the interior surface 217 of the handle body 214. Other connections such as snap-fit, friction fit, mating features, adhesives or welds may also be used for this purpose. As shown, a set screw 280 may be used to securely fasten the handle body nozzle 218 to the roller arm 220, wherein the set screw 280 passes through opening 282 defined in the handle nozzle body 218 extending inwardly through the exterior surface thereof. The proximal end 266 of the handle body nozzle 218 is also preferably of the same or a similar width and shape for a smooth transition between the handle body 214 and the handle body nozzle 218, although a rim or edge for facilitating removal as shown is within the scope of the invention or any enlarged design feature (shield, curve or shape provided for safety or aesthetic purposes). The distal end 264 of the handle body nozzle 218 preferably has a distal portion 258 that is configured and shaped to fit within a roller arm 220 in proximal end 221. As shown, the roller arm has a connector and attachment end 221 which can receive the distal portion 258 of the handle nozzle body and has a similar optional set screw receiving hole. An electrical male/female connector as described in embodiment 10 may be positioned within the end 221 and in the distal portion 258 of the handle nozzle body and/or between the distal end of the handle body 214 and the proximal end of the handle nozzle body for enabling electrical connectivity through the device or wires may be pulled through the device for connection to any heating element included.

In the embodiment shown, if electronic components are provided, the handle body nozzle 218 can be configured as shown in embodiment 10 to have within the distal portion 258 an electrical connector 56. Such connector can be of a variety of types of suitable electrical connectors as are known in the art, or to be developed, and, preferably interact with mating connectors provides electrical connection from a power supply (such as a battery or electrical wiring extending through the device to communicate with an electrical plug) to any electrical components within the handle body 214, roller head 240 or roller arm 220. As shown, the roller arm end 221 may incorporate such a connector as connector 22 described above in embodiment 10 and may have mating connection features such as receiving connector holes 23 on a first connector side 52 of a connector 52. It should be apparent that other connectors may be used or their positions reversed if desired.

When the handle body nozzle 218 is securely fastened to the roller arm if there are connectors therein, it would provides a secure electrical connection for the roller arm 220 and rolling head 240, which can also enable interchangeability of the roller head 240 alone or in combination with a roller arm 220 so that the entire distal combined portion of the device having the roller arm and roller head may be interchangeable if desired.

If electronic components are provided, a power supply preferably within the interior space of the handle body provides an electrical source and connection into the roller arm 220 and rolling head 240 components which are preferably each interchangeable alone or independently. Further, depending on the electronics provided if an electrical connection and heating element are provided, a sample wiring diagram showing the heating element 2130 the electronic controller 2134 (such as a programmable circuit, e.g., such as a microcontroller, raspberry pi and the like) can be configured for the type of electronics to be used. The battery or electric source can be used to apply a power source 2136 across the circuit. A sample circuit is shown as circuit 2132 in FIG. 17. One skilled in the art would know that the circuit and controls can be varied for temperature, time and other controls for heating the device and controlling the time and temperature, including any optional temperature sensors or alarms.

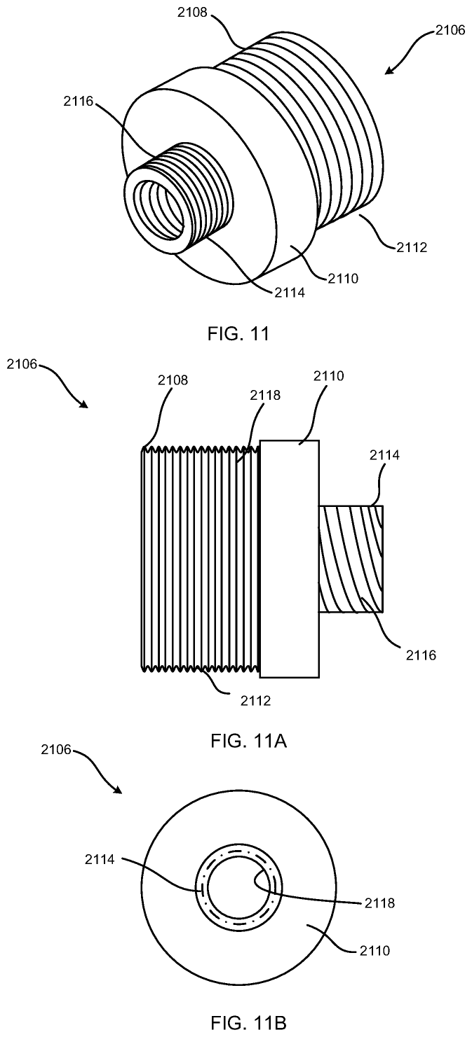

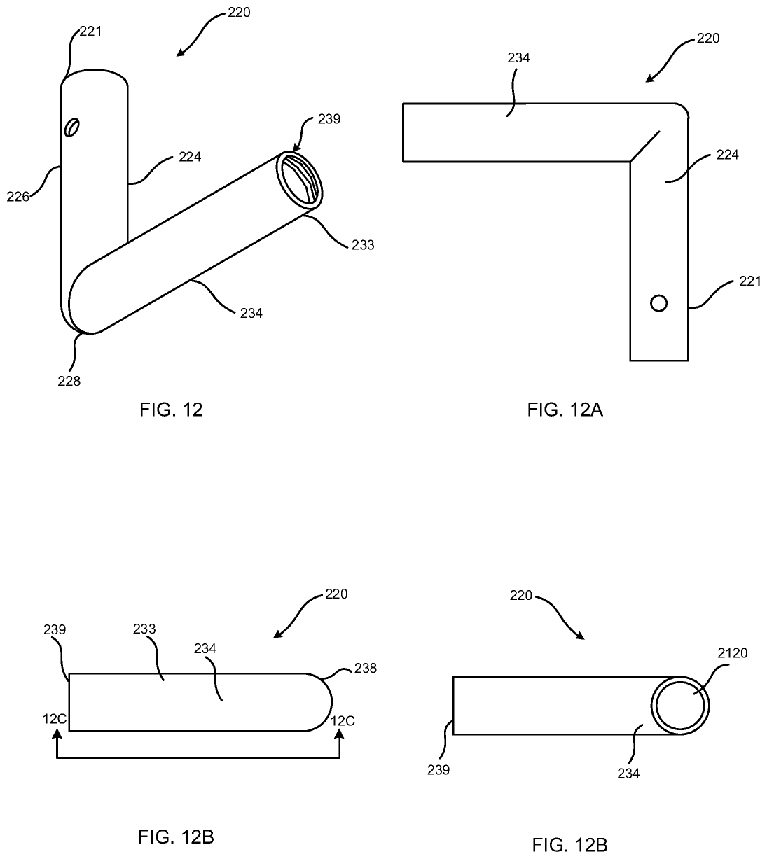

The roller arm 220 preferably includes an angled roller arm body 224 extending from end 221 (which may be a connector end) of the roller arm 220 to a mounting end 233. The roller arm body 224 may be designed in various shapes and configurations to suit aesthetics and/or utilitarian operation but is preferably configured to extend from the second end 221 and bend or curve to meet the roller head 240 so that the mounting end 233 connects to a roller end cap 2106. As shown in FIGS. 12-12C, threads on the interior at the mounting portion 239 at the end 233 end can receive mating threads 2108 on the roller end cap 2106. The roller end cap may include a snap fit that engages the mounting end 233, a friction fit, a rotatable cam or similar mechanical roller arm mountings. Such rotatable mountings are known in the art and may be modified as desired, with the threaded surfaces being preferred. The mounting end cap has a proximal end 2112 to engage the roller arm 220 and a distal end portion 2110 having an extension 2114 with preferred threads 2116 for engaging within the roller head 240 in a similarly sized bore 246 formed in the mating side 250 of the roller head.

It is preferred that the roller arm is formed of a material such as those mentioned above for use in forming the handle body. The roller head 240 may be solid with a portion that defines the receiving bore 246 for the roller arm 220 or hollow for incorporating desired electronic components as noted above or a support rod. As shown, the roller head ahs a hollow interior.

In a preferred embodiment as shown herein from the proximal end 221 to a first bend 228 to a first transversely extending portion 234 that runs from the bend 228 to the roller end cap 2106 which is then connected to the roller head 240. The mounting portion 339 of the roller arm 220 extends towards the roller head 40 from the bend 228 and connects with the roller end cap 2106. This is a simpler design than that noted above and has advantages when using electronics that extend up to or within the roller arm and/or roller head.

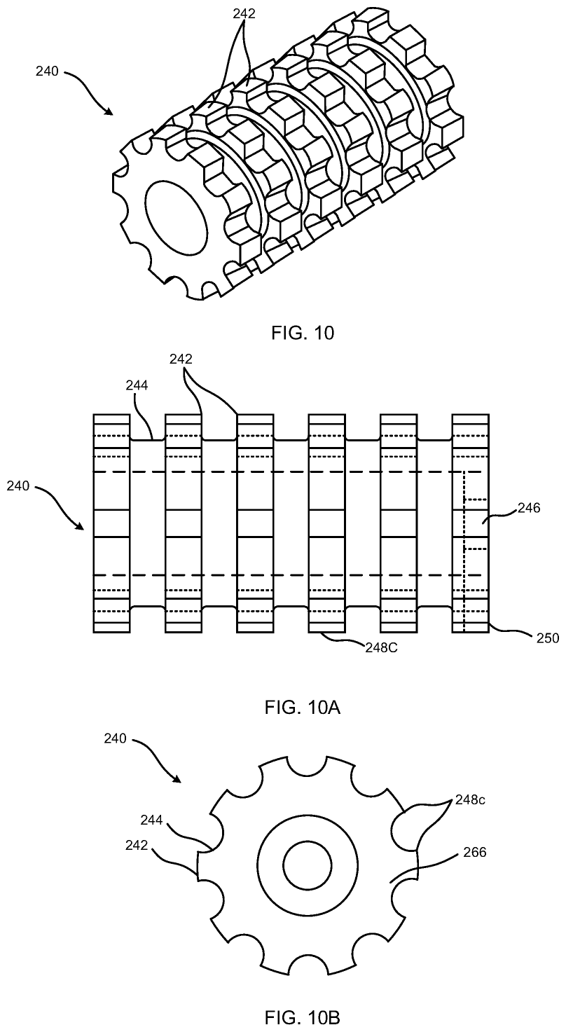

The rolling head 240 may be formed as a single unitary roller head body that is hollow (as noted above and as shown) or solid, either of which defines a mounting bore 246 for receiving a proximal end of the roller end cap 2106 as discussed above. The rolling head may be any of those described above. As shown in this embodiment, a roller head is formed as a series of parallel roller “wheels” each sitting in space apart side-by-side rows as shown in FIGS. 10-10B. Each “wheel” row has a series of protrusions 242 on its exterior surface 244. The shape of the protrusions 248c are blunt rectangular protrusions that slope downward to the surface 244. The protrusions as noted above may have various shapes and profiles but are preferably blunt and/or flat.

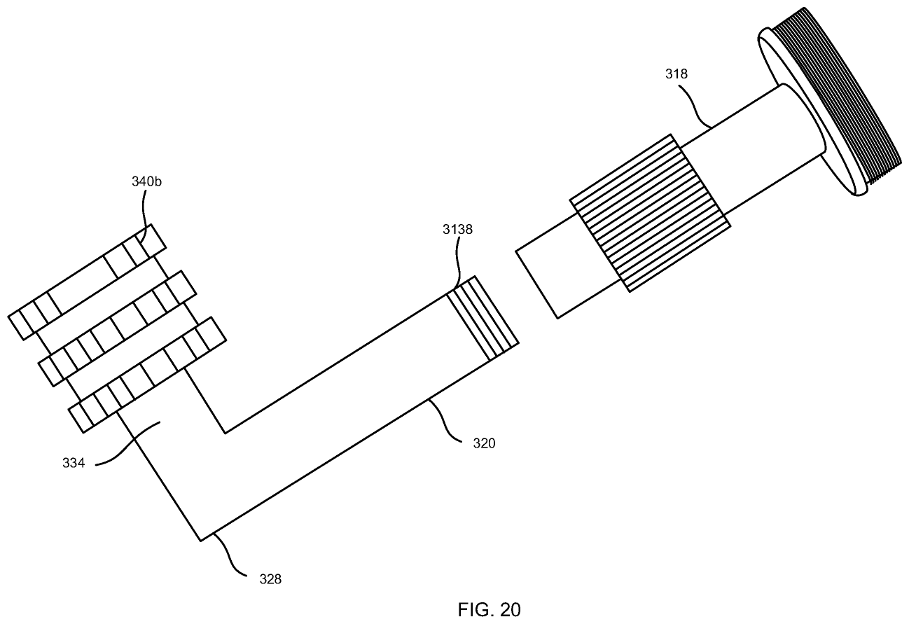

The protrusions 242 of the interchangeable roller heads may be offered in a variety of sizes (and shapes) to meet specific patient needs and to suit the particular barrier product being employed. Further the roller head length and number of “wheels” rows in series can be alternated for different end uses, such as smaller for tight areas or smaller wound treatments. This is shown in various embodiments 300 shown in FIGS. 18-20. In FIG. 18, a single row of protrusions on a roller 340a is shown attached directly (although it may be attached indirectly such as through a roller end cap as described in embodiment 210) to a roller arm 320 on a distal arm 334 and a longitudinal proximal arm 320. On the proximal end, optional screw threads 3138 are provided for receiving a twist screw connector 3140 as shown in FIGS. 19 and 20 which may be fitted on the nozzle head body 318. The twist screw connector 3140 includes interior mating threads to engage the threads 3138. In FIG. 20, a three row roller head 340b is shown as a two-row roller head 340c is shown in FIG. 19.

The roller head and its protrusions may be formed of the same materials noted above, including surgical stainless steel, aluminum, titanium and other metals and their alloys, polymer and composite materials, composites and other suitable materials as note above. The protrusions herein can gently assist in successfully obtaining a leak-proof mold and seal around the actual stoma or other wound area by pressing the innermost edge of the barrier compound seal, or the collar thereof circumferentially until a full rotation is made around the edge of the wound and the barrier compound is indented or pressed by flat tip head, securing and increasing the border edge integrity between the compound and the wound.

The of embodiments 10, 210 and 300 may be provided alone or in a kit. For convenience purposes a kit 100 as shown schematically in FIG. 2, is described with respect to embodiment 10 (although it will be understood based on this disclosure that all embodiments may be in a kit) that includes one or more of the medical device as described above in detail, having a handle such as handle 12, a roller arm, such as roller arm 20 and a roller head 40 having an exterior surface 44 on the roller head 40 configured to enhance adhesion of a barrier product such as BP in FIG. 1 to an area A of mammal skin MS near a wound opening. The kit 100 preferably also includes instructions 102 describing how to use the medical device 10. If the medical device 10 is formed so as to have interchangeable roller heads (or roller head and roller arm combinations), one or more additional such interchangeable roller heads and/or roller arms may be provided to the kit. A barrier product BP as described herein may also be incorporated into the kit 100 such as the barrier products described hereinabove. Such barrier products may include a barrier and a barrier compound as described also above. A carrier case or other suitable container may be provided to the kit as an optional feature (not shown).

The invention also includes a method of applying a barrier product for wound care. The method will also be described herein with respect to embodiment 10 for convenience purposes, but can be practiced with any of the embodiments herein. The method includes providing a medical device such as the medical device 10 described herein in its various embodiments having a handle 12 as noted above, a roller arm such as roller arm 20 and a roller head such as roller head 40 having an exterior surface 44 thereon configured to enhance adhesion of the barrier product to an area of mammal skin near a wound opening.

The barrier product is then applied to an area of mammal skin near a wound opening so as to adhesively contact the area of the mammal skin near the wound area. The mammal skin may be the skin of a human or an animal requiring wound care treatment. The wound area may be the area near or around the wound, i.e., the peri-wound area. The wound may be a fistula, stoma or other open wound as described above.

The roller head 40 is manually contacted to the barrier product BP and then the roller head 40 in one of its interchangeable forms and having protrusions 42 thereon is rolled across the barrier product BP in the area A of the mammal skin MS near a wound. The roller had 40 includes blunt protrusions 42 as described above on the exterior surface 44 of the roller head 40 which protrusions contact the barrier product when the roller head is moved.

The mammal skin is preferably human skin, the area near the wound is preferably a pen-wound area, and the barrier product preferably includes a barrier compound. The method can include applying the barrier compound to the pen-wound area and then rolling the roller head around the pen-wound area. In the method, the roller head 40 may be rolled in a one-way direction where it is rolled, lifted and placed at another starting point and then rolled again in a one-way direction, or may be rolled in back-and-forth motions or may be rolled in a circular direction, or some combinations thereof

The medical device 10 may include the heating element or other electronic components as noted above and the method may further include heating the roller head 40 prior to contacting the roller head to the barrier product or during the rolling process to heat the barrier product and/or skin surface to skin safe temperatures.

Also included is a method for increasing adhesion of a barrier product to skin of a mammal in an area near a wound. The method includes providing a medical device as described herein such as medical device 10 and applying the barrier product BP to an area A of mammal skin MS near a wound opening so as to adhesively contact the area A of the mammal skin MS near the wound area. The roller head 40 is contacted to the barrier product BP and the roller head 40 is rolled across the barrier product BP in the area A of the mammal skin MS near the wound area.

Other options for consideration in the invention herein include a rubberized sleeve on the handle body for comfort of the user in use. Various packaging for selling the devices as single products in single product packaging, with components as kits, in multiple size, color and shape choices, and variations of the components in length, width, and shape may also be made. The size and arrangement (whether staggered or aligned in rows) on the roller head of the protrusions to be blunted as curved nubs, rivets or other shape may also be varied in product sales and options. The use of electronic components including a heating element are also available for optional embodiments of the medical device and/or a kit including the device.

If the medical device has releasably detachable components, the components and any interchangeable versions thereof may be packaged independently or together or the device sold as a single device having a unitarily formed handle and handle nozzle with optionally a unitarily formed roller arm as discussed above. It is also possible provide a case or other container for the medical device in which the capability is provided to heat the entire case or container (not shown), resulting in an alternative method to heat the medical device or the case or container may be configured to allow heating by other methods such as submersion in heated water, insertion of the case or container into a separate, heating mechanism (e.g., an oven, microwave oven, or the like). The medical device or its components may also be heated by way of removing the device and/or its components its packaging, case or container and using such heating sources and devices to separately warm or heat the medical device or its components, e.g. a stand-alone heating source, a warming box, a warming pouch or the like

For options wherein electronic components are provided, in addition to heating elements, the roller head may be wired to be electrically rotatable by using a power source (wire to a plug, cordless chargeable battery, replaceable battery, solar cell, and the like to power a rotatable motor to rotate the roller head while manually pushing the device, but allowing the roller head to turn on its own. way of cordless electrical operation. Such device can also include a remote control option for heating or activating the roller mechanism.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A medical device, comprising
a handle;
a roller arm;
and a roller head, wherein an exterior surface of the roller head is configured to enhance adhesion of a barrier product to an area of mammal skin near a wound opening.

2. The medical device according to claim 1, wherein the handle comprises:
a handle body having a proximal end and a distal end;
a handle nozzle on the distal end of the handle body, wherein the handle nozzle is configured to receive a roller arm attachment on the roller arm.

3. The medical device according to claim 2, wherein the roller arm attachment is positioned on a proximal end of the roller arm.

4. The medical device according to claim 2, wherein the handle body and the handle nozzle are releasable attachable.

5. The medical device according to claim 2, wherein the handle body defines a hollow interior space for receiving wiring and/or electronics.

6. The medical device according to claim 5, Wherein the handle body has a removable inner sleeve surrounding the interior space.

7. The medical device according to claim 5, wherein the proximal end of the handle body has a removable end cap thereon.

8. The medical device according to claim 2, wherein the roller arm attachment comprises an electrical connector.

9. The medical device according to claim 8, wherein the handle body defines a hollow interior space for receiving wiring and/or electronics, and the electrical connector is a female connector configured to receive a male connector in electrical communication with the electronics positioned within the hollow interior space of the handle body.

10. The medical device according to claim 9, wherein the electronics are one or more of a heating element, a power source, a lighting element, and/or a motor.

11. The medical device according to claim 2, wherein the handle nozzle has a proximal end configured to connect to the distal end of the handle body, and the handle nozzle has a distal end having a distal end portion configured to mate with the attachment on the roller arm, wherein the distal end of the handle nozzle further comprises an electrical connector for engaging an electrical connector on the attachment on the roller arm.

12. The medical device according to claim 11, wherein the electrical connector on the distal end of the handle nozzle is a male connector and the electrical connector on the attachment on the roller arm is a female connector.

13. The medical device according to claim 1, wherein a proximal end of the handle has a removable end cap thereon.

14. The medical device according to claim 1, wherein the handle has an exterior surface configured for manual gripping.

15. The medical device according to claim 1, wherein the roller arm comprises:
an angled roller arm body extending from a connector end to a mounting end.

16. The medical device according to claim 15, wherein the roller arm body is configured to extend from the connector end and bend around the roller head so that the mounting end fits within an opening in the roller head.

17. The medical device according to claim 16, wherein the roller arm body comprises connector configured to mate with the handle nozzle on a first side of the connector and to engage a first longitudinally extending portion on a second side of the connector, a first bend extending between the first longitudinally extending portion and an angled portion, a second bend extending between the angled portion and a first transverse portion, a third bend between the first transverse portion and a second angled portion; and a fourth bend extending between the second angled portion and a mounting portion.

18. The medical device according to claim 1, wherein the handle body and the handle nozzle are formed as a unitary single piece body.

19. The medical device according to claim 1, wherein the roller head comprises protrusions arranged in a staggered pattern on the exterior surface of the roller head.

20. The medical device according to claim 1, wherein the roller head comprises protrusions extending from the exterior surface of the roller head and having a blunt tip.

21. The medical device according to claim 1, wherein the roller head comprises protrusions extending from the exterior surface of the roller head and having a rivet-shaped tip.

22. The medical device according to claim 1, wherein the roller head has an opening on at least one side thereof for receiving a mounting portion of the roller arm.

23. The medical device according to claim 22, wherein the roller head is removable from the roller arm.

24. A medical device kit, comprising
a medical device, comprising
a handle;
a roller arm; and
a roller head, wherein an exterior surface of the roller head is configured to enhance adhesion of a barrier product to an area of mammal skin near a wound opening; and
instructions for using the medical device.

25.-27. (canceled)

28. A method of applying a barrier product for wound care and/or for increasing adhesion of a barrier product to skin of a mammal in an area near a wound, comprising:
(a) providing a medical device, comprising
a handle;
a roller arm; and
a roller head, wherein an exterior surface of the roller head is configured to enhance adhesion of a barrier product to an area of mammal skin near a wound opening;
(b) applying the barrier product to an area of mammal skin near a wound opening so as to adhesively contact the area of the mammal skin near the wound area;
(c) manually contacting the roller head to the barrier product and rolling the roller head across the barrier product in the area of the mammal skin near the wound area.

29.-35. (canceled)
